(19) United States
(12) Patent Application Publication
(10) Pub. No.: US 2004/0009610 A1
(43) Pub. Date: Jan. 15, 2004

(54) Organic contaminant soil extraction system

(75) Inventors: John F. Schabron, Laramie, WY (US); Susan S. Sorini-Wong, Laramie, WY (US)

Correspondence Address: SANTANGELO LAW OFFICES, P.C., 125 SOUTH HOWES, THIRD FLOOR, FORT COLLINS, CO, 80521, US

(73) Assignee: The University of Wyoming Research Corporation d/b/a Western Research Institute, Laramie, WY

(21) Appl. No.: 10/617,247

(22) Filed: Jul. 9, 2003

Related U.S. Application Data
(63) Continuation-in-part of application No. 09/558,979, filed on Apr. 27, 2000.
(60) Provisional application No. 60/131,648, filed on Apr. 29, 1999.

(51) Int. Cl. G01N 33/24; G01N 1/38

(52) U.S. Cl. 436/178; 73/866; 436/174; 422/68.1

(57) ABSTRACT

A device and method for testing a soil sample for organic contaminants, including stirring soil samples and solvent for subsequent testing of an extracted phase with a plurality of interchangeable rotationally configured stir implements for testing of a variety of soils.

This application is a continuation-in-part of U.S. application Ser. No. 09/558,979 filed Apr. 27, 2000, which claims the benefit of U.S. Provisional Application No. 60/131,648, filed Apr. 29, 1999, both hereby incorporated by reference.

### I. ACKNOWLEDGMENT OF GOVERNMENT SUPPORT

This invention was made with federal government support under Cooperative Agreement Number DE-FC21-93MC30127 awarded by the U.S. Department of Energy. The federal government may have certain rights in this invention.

### II. BACKGROUND OF THE INVENTION

Generally, this invention relates to a soil testing device and screening techniques which may be utilized for determining various contaminates in soil. Specifically, the invention focuses upon the devices and methods that may be used to provide an improved technique for efficiently stirring soil and extraction solvent for consistent extraction and the analysis of organic contaminants in soil. The device may be used either in the laboratory or as a field-portable unit.

Contamination of the earth by organic contaminants is a major concern due to the possible environmental, health and financial problems relating to such contamination. Possible contaminants include a variety of organic materials such as crude petroleum, fossil fuels, lubricating oils and greases, solvents and others. Other contaminants may include inorganic, metals, ions and the like. It is economically and socially desirable to be able to identify contaminated sites so that potential risks can be evaluated and remedial action can be properly conducted.

Sophisticated laboratory techniques are available to measure the presence and level of contaminations in earth samples. Laboratory techniques, however, are notoriously expensive and time consuming. Furthermore, field analysis typically requires many tests and portable instruments to perform the analysis. It is, therefore, desirable to have a testing technique which could be easily and inexpensively used for field testing to identify contaminated soils.

Other field testing techniques have been proposed in which a soil sample is extracted with a solvent to remove organic contaminants. The extracted phase may then be analyzed to gain information concerning the presence of contaminants in the soil sample and the level of contamination at a particular location. In particular, and as discussed in U.S. Pat. No. 5,561,065, hereby incorporated by reference, extraction may be performed in the field by stirring an earth sample with a solvent in a suitable container, such as a glass jar. The solvent and the earth sample may be thoroughly mixed by a mechanical mixer to assure proper contact between the sample and the solvent. Subsequently, the extracted phase may be separated from the solid residue using known separation methods and the extracted phase may be analyzed for contaminants.

Yet, prior soil testing techniques may have limited testing to specific types of soils. In the past, testing devices may not have been adequately functional for testing on soils that were composed of rocks, plant material, or other substances. Before a soil sample could be tested, it may have been necessary to remove the rocks or other undesirable components from the test sample. This may have been time consuming and may even have affected the results of the test.

During the course of the present inventors' work to develop a field test kit to determine diesel contamination in soils by extraction, the primary means of stirring the soils and solvent was magnetic stirring. In the laboratory this seemed an optimal approach, but for field use where powerful 115-volt laboratory stirrers are not always available, the small commercially available portable magnetic stirrers tested did not always have enough torque to spin the stir bar, or magnetic contact may have been lost between the stir bar and the motor mounted magnet. Other field test kits such as the immunoassay test kits use hand-shaking systems for soil extraction, which can result in inconsistent extractions and possible wrist injury with repetitive extractions.

However, the prior techniques and devices have not always addressed adequately the known problems associated with prior stirring devices. Prior techniques did not always adequately address providing a test that allows efficient and repeatable results. In particular, the stirring devices often available could not adequately stir a soil and solvent mixture with different types of soils. For example, soils that include small rocks or plant material may be difficult to stir and effectively test. Furthermore, prior devices may not have adequately addressed the need for sealing the electrical and motorized components of a stirrer from volatile vapors generated during the sampling and stirring process. The presence of ignitable vapors in solvents and soil samples can create a high risk of an explosion or fire during the stirring process. Also, the small stirring devices often available could not adequately maintain the torque required to adequately stir a soil and solvent mixture. Field stirrers may not have maintained proper coupling between the motor and stirrer during varying levels of torque. Additionally, field stirrers previously lacked appropriate transportability and flexibility for field decontamination procedures prior to a subsequent use. Therefore, a need has been discovered for a field stirring device and method that could be used in a variety of soil types and provide transportability and flexibility features that allow for ease of use in the field and for necessary decontamination procedures.

### III. SUMMARY OF THE INVENTION

The present invention provides a field test device that practically provides not only for a proper amount of stirring, but that also practically provides for the needs of:

1) effectively stirring a variety of different types of soil;

2) maintaining constant coupling between a motor and a stirring element;

3) sealing the motorized components and outer regions from volatile gases present in the sample container;

4) providing transportability and flexibility in a field test device; and

5) providing ease in decontamination procedures.

Accordingly, the present invention provides a soil test device and method for stirring soil samples and solvent and subsequent testing of an extracted phase. The present invention may even be considered, in some aspects, as a development away from that which was previously known in the art of soil contamination sampling.

It is an object, therefore, of the present invention to provide a soil testing device and method for use in determining the extent of organic, chemical, or fuel contamination in soil.

In particular, it is an object of the invention to provide a soil testing device and method that can be used with a variety of soils.

It is an object of the present invention to provide a soil testing device and method for testing soil samples that achieves the proper amount of stirring of soil samples and extraction solvent for soil analysis.

A further object of the present invention is to provide a soil testing device and method for testing soil samples that allows for the sealing of motorized components of the device and outer regions thereof from volatile gases present in the sample container.

It is also an object of the present invention to provide a soil testing device and method for testing soil samples that allows for the retention of coupling between the stirring element and the motor such that a continuous coupling will be retained during sample and extraction solvent stirring.

Additionally, it is an object of the present invention to provide a soil testing device and method for stirring soil samples and solvents that retains the optimal field characteristics of transportability and flexibility and that allows for ease of use in the field and for necessary decontamination procedures.

Other objects of the invention are disclosed throughout other areas of the specification and claims. In addition, the goals and objectives may apply either in dependent or independent fashion to a variety of other goals and objectives in a variety of embodiments.

### IV. BRIEF DESCIPTION OF THE DRAWINGS

FIG. 1 is a side view of an embodiment of the invention showing a soil stirring and testing device, including that of external controls and power supply connections for the device.

FIG. 2 is a cross sectional view of the embodiment shown in FIG. 1, wherein the external connections and controls are omitted for simplicity.

FIG. 3a is a top view of an embodiment of the invention of an interchangeable implement.

FIG. 3b is a side view of an embodiment of the invention of an interchangeable implement.

FIG. 4a is a top view of an embodiment of the invention of an interchangeable implement.

FIG. 4b is a side view of an embodiment of the invention of an interchangeable implement.

FIG. 5a is a top view of an embodiment of the invention of an interchangeable implement.

FIG. 5b is a side view of an embodiment of the invention of an interchangeable implement.

### V. DETAILED DESCRIPTION OF THE PREFERRED EMBODIMENTS

As mentioned earlier, the present invention includes a variety of aspects, which may be combined in different ways. The following descriptions are provided to list elements and describe some of the embodiments of the present invention. These elements are listed with initial embodiments, however, it should be understood that they may be combined in any manner and in any number to create additional embodiments. The variously described examples and preferred embodiments should not be construed to limit the present invention to only the explicitly described systems, techniques, and applications. Further, this description should further be understood to support and encompass descriptions and claims of all the various embodiments, systems, techniques, methods, devices, and applications with any number of the disclosed elements, with each element alone, and also with any and all various permutations and combinations of all elements in this or any subsequent application.

As can be understood from the drawings, the basic concepts of the present invention may be embodied in different ways. The present invention, in embodiments, may provide methods and devices for determining if organic contaminants are present in soil. Initially, a soil sample may be selected from a potentially contaminated site. Naturally, soils may vary in composition. For example, some soils have rocks, small rocks, plant material, stringy plant material, among other things. A soil sample may include a combination of various components.

In embodiments, the present invention tests soil samples for various types of organic contaminants. Of course, the invention may include testing soils for organic, inorganic, metals, ions, and the like. For example, the invention may include, but is not limited to, testing soils for sodium, calcium, magnesium, iron, copper, nitrate, sulfate, phosphate, nutrients, or metallic contaminants, such as, but not limited to arsenic, selenium, lead, mercury, and the like. In particular, as but one example, the invention may test soils for organic contaminants, such as fuel contaminants. Fuel contaminants may include, but are not limited to, diesel, tar, motor oil, fuel oil, petroleum, coal tar, gasoline, aviation gasoline, synthetic motor oil, and the like.

Further, the present invention may provide for selecting a contaminant test substance that may be suitable with the type of soil sample selected for that particular test. In an embodiment, a contaminant test substance may be a substance that may chemically react with a contaminant, or even an organic contaminant and allow detection and extraction of such contaminant. Depending on the composition of the soil sample, different organic contaminant test substances may work better. It is noted that the type of test substance that would work best with the particular components and contaminants in a soil sample is known to those skilled in the art.

In embodiments, an organic contaminant test substance may be an organic solvent and may even be an aqueous solvent, or the like. Of course, any type of organic, aqueous, or water based solvent, such as acid or base and the like may be used. Some examples of these may include, but are not limited to, alcohols, aliphatic hydrocarbons, aromatic hydrocarbons, chlorinated solvents, ketones, esters, amines, ethers, sulfides, carboxylic acids, surfactant solutions, and like solvents.

In order to achieve a desired soil testing sample, the present invention may include, in an embodiment, adding an organic contaminant test substance to a soil sample to create a soil testing sample. Further, the soil testing sample may be put into a container. This may be a cylindrical container (1). Of course, this may include, in an embodiment, putting a soil sample in a container as well as putting an organic contaminant test substance in a container, which ultimately creates a soil testing sample.

In an embodiment, the present invention may include the addition of a drying agent to the soil sample. As but one example, calcium oxide may be added to a soil sample. The drying agent may dry the soil and may even bind to materials in the soil sample to minimize interference. In other embodiments, the invention may include screening a soil sample to remove larger items from the soil sample. This may include large items, such as rocks, branches, debris, or the like that may prevent the appropriate stirring of the sample.

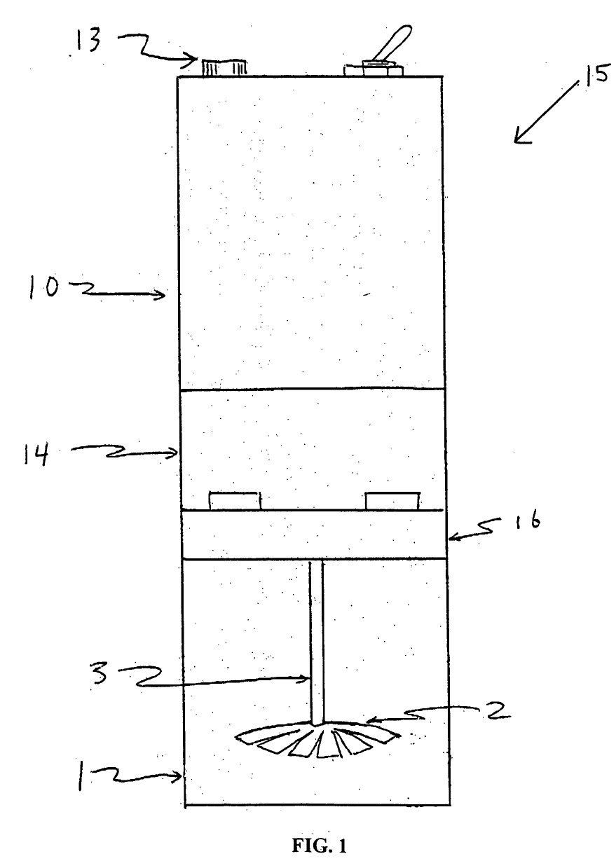

FIG. 1 shows a perspective view of one embodiment of the soil stirring and testing device (15) of the present invention. The present invention may include enclosing a soil testing sample in a cylindrical container (1). In order to achieve this, the device may comprise a cylindrical container (1), having an opening (not depicted) covered by a cylindrical removable enclosing element (16). In embodiments, a container and removable enclosing element may be any shape, such as, but not limited to an oval, circle, square, and the like shapes. Further, the cylindrical enclosing element (16) may be configured to cover the opening of the cylindrical container (1). This may include designing the cylindrical enclosing element (16) to securely attach and close the opening of the cylindrical container. For example, the enclosing element may screw on or may even be clamped to the container. Of course, the enclosing element may be opened or removed from the container. Moreover, this may include designing an enclosing element to fit on any shape container.

In embodiments, the cylindrical removable enclosing element (16) may provide housing for a motor (5). Naturally, the enclosing element (16) may provide housing for other components, including but not limited to a motor, clutch assembly, magnetic clutch assembly and/or other components.

The cylindrical container (1) may be a made of polypropylene, glass, polyphenylene sulfide, polyethylene, chlorofluorocarbon or teflon polymer, polyphthalamide, or similar materials. Also, in an embodiment, the cylindrical removable enclosing element (16) may be made of polypropylene, glass, polyphenylene sulfide, polyethylene, chlorofluorocarbon or teflon polymer, polyphthalamide, or other materials. For example, the cylindrical removable enclosing element (16) may even be a jar cover designed to contain the contents of the container. Accordingly, the invention may provide for enclosing a soil testing sample in a cylindrical container with a jar cover. The enclosing element, however, need not be a separate element, as described below.

A significant aspect of an embodiment of the present invention may be the use of a plurality of interchangeable rotationally configured stir implements (2). As described in an object of the invention, it is desirable to have a device that can be used with different types of soils. Suitably, the present invention may include axially restraining a plurality of interchangeable rotationally configured stir implements (2) with a shaft element (3) in a cylindrical container (1). In the field, it may be more convenient to be equipped with different stir implements to test a variety of different kinds of soil samples. The present invention may provide, in embodiments, detachably connecting a plurality of rotationally configured stir implements (2) to a shaft element (3). Depending on the soil sample components, there may be a preferable implement that effectively stirs that soil sample. Specifically, an embodiment may include providing an implement that may be attached to a shaft element (3), which, when in use may rotate on an axis. This may result in a movement that causes stirring of a soil sample.

In order to securely connect the implements (2) to a shaft element (3), the present invention may include providing a multi-directional restraint (4). This may allow the implement to be firmly attached to a shaft element (3). Appropriately, the present invention may provide a multi-directional restraint (4) to which an interchangeable rotationally configured stir implement (2) may be responsive. The implement (2) may be connected to a shaft with a user detachable connection element, including, but not limited to, a screw, fastener, clamp, and the like element.

The shaft element (3) and interchangeable rotationally configured stir implements (2) may be made from a nonreactive material such as stainless steel and may be positioned within a container and designed to be moved in a manner resulting in the stirring or combining of the contents of the container. According to an embodiment, the stir implement (2) may be located above the level of soil testing sample within a container. In particular, stirring may be performed above the soil level in the container. Stirring above the soil level may result in favorable extraction of contamination from the soil sample.

The present invention may include stirring a soil testing sample which may cause common axial displacement by a plurality of interchangeable rotationally configured stir implements (2) to create an extraction sample. In order to achieve the desired stirring, the present invention may include divergently displacing a soil testing sample with one of the interchangeable rotationally configured stir implements. For example, the present invention may include divergently displacing a soil testing sample with an angularly displaced circular element (6), or even divergently displacing a soil testing sample with an angularly displaced linear element (7). In another embodiment, the present invention may include shearing a soil testing sample with one of the interchangeable rotationally configured stir implements. For example, shearing a soil testing sample may occur when using an angularly displaced circular element (6). Further, in such a manner, it may be important to gently displace a soil testing sample with one of the interchangeable rotationally configured stir implements. For example, if a soil sample contains plant material or even stringy plant material, it may be desirable to gently displace the sample with one of the interchangeable rotationally configured stir implements. Particularly, in an embodiment, the present invention may include gently displacing stringy plant material with a round ended bar element (8). Moreover, if a soil sample contains small rocks, the present invention may include displacing the soil testing sample with an angularly displaced linear element (7). Indeed, other combinations and permutations may be appropriate.

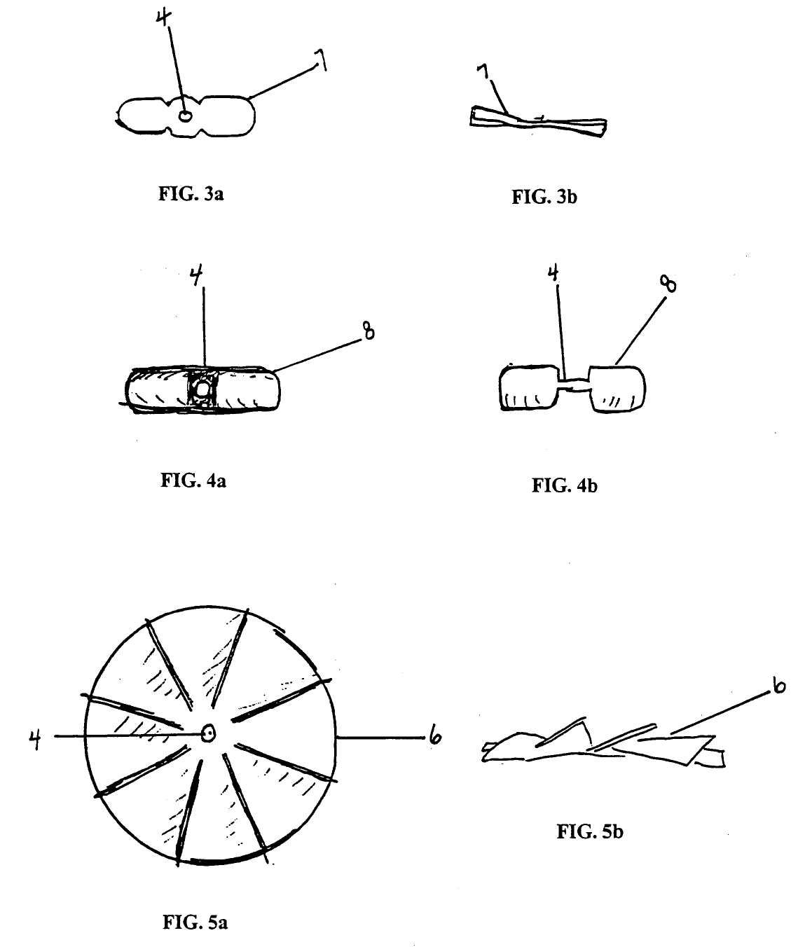

In embodiments and as shown in FIGS. 3a, 3b, 4a, 4b, 5a, and 5b, an interchangeable rotationally configured stir implement (2) may represent a round ended bar element (8), an angularly displaced circular element (6), an angularly displaced linear element (7), a divergent displacer element, a gentle displacer element, or any other type of stir element.

When selecting an interchangeable rotationally configured stir implement (2), it may be important to select the desired implement from a combination of various implements with particular types of soil components. Such possibilities may include:

a round ended bar element (8) used with plant material,

a round ended bar element (8) used with stringy plant material,

a divergent displacer element used with small rocks,

a gentle displacer element used with plant material,

a gentle displacer element used with stringy plant material, and

an angularly displaced linear element (7) used with small rocks.

Though these are merely examples of combinations of stir implements with a type of soil, it may be desirable to use different combinations and it may even be desirable to use a particular type of implement when a soil sample includes several different components. Such as, it may be more desirable to use a divergent displacer element when a soil sample contains both rocks and stringy plant material.

In embodiments, the present invention may provide for rotating a shaft element (3) which causes the motion for stirring. To effectively stir a soil sample, it may be appropriate to continuously rotate a shaft element (3). In order to rotate the interchangeable rotationally configured stir implement (2) and shaft element (3), as discussed earlier, the present invention may provide for a motor (5) and may even provide for a clutch assembly (14). Accordingly, a shaft element (3) may rotate when a motor (5) is in operation and thus may be responsive to a motor (5).

In embodiments, the present invention may include powering a motor (5). Accordingly, a power source may be connected to a motor (5) with a power source connection (13). This power source may include a battery, among other power elements. As such, a shaft element (3) and motor (5) may be responsive to the power source.

In embodiments, the present invention may provide for a clutch assembly (14) to which a shaft element (3) may be responsive. This clutch assembly (14) may be powered by a motor (5) and may allow the shaft element (3) to rotate. In an embodiment, clutch assembly (14) may be a magnetic clutch assembly. The magnetic clutch assembly may magnetically couple the shaft element (3) to the motor (5), so as to provide engagement and stirring movement of the interchangeable rotationally configured stir implements (2) during operation of the test device and operational movement of the motor (5). A mechanical or other barrier may exist between the motor (5) and shaft element (3), formed by the magnetic clutch assembly, such that vapors within the container may be isolated from the motor (5) within an encasing (10).

Importantly, it may be appropriate in some embodiments to provide a seal between the cylindrical container (1) and the motor (5). The inclusion of the magnetic clutch assembly and its orientation relative to the motor (5) and container may even provide a vapor seal. Accordingly, the present invention may include, in embodiments sealing a soil testing sample from a motor (5), and may even include magnetically sealing a soil testing sample from a motor (5). Furthermore, an embodiment may include a shaft element (3) which may go through a cylindrical removable enclosing element (16). Specifically, a seal (11) may be interposed adjacent to the shaft element (3) and cylindrical removable enclosing element (16).

In embodiments, an encasing (10) may be removably connected to the enclosing element (16), wherein the encasing (10) may house operable elements for the testing device (15). For example the encasing (10) may include a motor (5). Operable controls and a power source connection (13) may be provided on the encasing (10) to provide electrical power and control for testing operations. Open spaces may also be provided in the encasing (10) to facilitate rinsing and other decontamination procedures. In an embodiment, the present invention may provide for encasing at least a motor (5) in a cylindrical housing. In other embodiments, an encasing (10) may include, but is not limited to a polypropylene encasing, glass encasing, polyphenylene sulfide encasing, polyethylene encasing, chlorofluorocarbon polymer encasing, polyphthalate encasing, and the like.

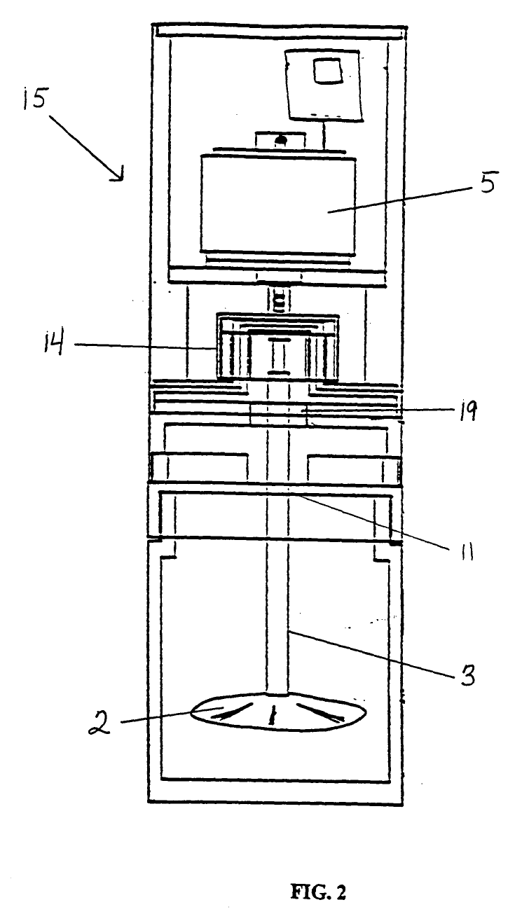

FIG. 2 depicts a cross sectional view along the axial length of a test device embodiment shown in FIG. 1. In FIG. 2, an encasing (10) may be located around the motor (5). In other embodiments, an encasing (10) may include either or both a motor (5) and a clutch assembly (14). The clutch assembly (14) may be positioned adjacent a cylindrical removable enclosing element (16) and may even be removably attached to the enclosing element (16) by way of the connection of the encasing (10) to the enclosing element (16), as depicted and previously described. In an embodiment, the motor (5) may be removably attached to the enclosing element (16) by way of the connection of the encasing (10) and clutch assembly (14) to the enclosing element (16). The motor (5) and shaft element (3) may be coupled to the clutch assembly (14). An embodiment of the invention, however, may provide that a portion of the clutch assembly (14), for example, the bottom of the clutch assembly (14) may serve as the enclosing element. Therefore, the enclosing element need not be a separate element, apart from the other elements of the stirring mechanism. Of course, the enclosing element may be a separate element, as disclosed herein. Further, the encasing (10) and cylindrical removable enclosing element may be one piece, but it is not limited to a single piece. During operation of the test device, and in response to operative movement by the motor (5), the clutch assembly (14) may function to provide movement of the shaft element (3) and interchangeable rotationally configured stir implements (2). In an embodiment, the shaft element (3) may be provided with a bearing (19) to facilitate the stirring motion induced by the motor (5) via the clutch assembly (14).

When a soil sample has been stirred using the present invention's devices and methods, an extraction sample may be taken from the stirred soil testing sample. In an embodiment, the present invention may provide for analyzing an extraction sample to determine the presence of an organic contaminant, among other contaminants. In an embodiment, an analysis element may be used to test the extraction sample for an organic contaminant. This analysis element may include, but is not limited to a UV or visible absorption spectroscopy, fluorescence or raman spectroscopy, gas chromatography, mass spectrometry, atomic fluorescence, atomic absorption, atomic emission, electrochemistry, colorimetric reactions, titration, gravimetric analysis, and those otherwise known to those skilled in the art. Accordingly, the present invention may provide for analyzing a soil sample to determine an organic contaminant including, but not limited to diesel, tar, motor oil, fuel oil, petroleum, coal tar, gasoline, aviation gasoline, synthetic motor oil, and the like.

In an embodiment, and as depicted in FIGS. 1 and 2, the motor (5) and the clutch assembly (14), by way of the encasing (10), may be arranged in a top and middle orientation with respect to a bottom orientation of the container. This orientation may provide for a coaxial arrangement of the magnetic clutch assembly (14), enclosing element and cylindrical container (1) during operation. Furthermore, and in accordance with an embodiment of the invention, the motor (5), the clutch assembly (14) and the container may be cylindrical in shape. The shape and orientation of the particular elements may provide an arrangement that enhances, and indeed comprises in part, inventive aspects of the invention. In particular, the shape and orientation of the motor (5), the clutch assembly (14) and the container may ensure the proper sealing function and stirring ability of the device, as described herein. Thus, the shape and orientation of the motor (5), the clutch assembly (14), and the container may provide a testing device that isolates vapors contained within the container from the motor (5).

In other embodiments, the present invention may include a cylindrically aligned exterior, as mentioned above. This exterior may include a cylindrical removable enclosing element (16), a cylindrical container (1), and a magnetic clutch assembly. Accordingly, the present invention may provide, in embodiments, cylindrically aligning an exterior of a cylindrical container (1), an enclosing element (16), and a cylindrical encasing. In other embodiments, the motor (5), magnetic clutch assembly, and cylindrical container (1) may comprise a top, middle, and bottom arrangement, respectively. Further, in embodiments, the encasing, cylindrical removable enclosing element (16), and cylindrical container (1) may comprise a cylindrically aligned exterior.

According to an embodiment, the invention may be accomplished and practiced by a method of testing a soil sample. One method provides the device as described above and includes the operational engagement of the motor (5) and the interchangeable rotationally configured stir implements (2). A soil sample and extraction liquid or organic contaminant test substance may first be inserted within the cylindrical container (1) via the container opening and the container may be at least partially closed via the enclosing element (16) or jar cover. The motor (5), clutch assembly (14) and encasing (10) may be attached to the enclosing element (16). The device may then be operated by the engagement of the motor (5) and the interchangeable rotationally configured stir implements (2) by way of the clutch assembly (14). Operation of the device, therefore, may provide a stirring motion of the shaft and interchangeable rotationally configured stir implement (2) which may allow for stirring of the soil sample and organic contaminant test substance. For example, an embodiment, and not by way of limitation, comprises affecting a rotation of the shaft element (3) and interchangeable rotationally configured stir implement (2) by the operable engagement with the motor (5), via the clutch assembly (14). Subsequently, and after a sufficient amount of stirring, the soil sample, per the extraction phase or extraction sample, may be tested for contamination.

The motor (5), clutch assembly (14) and container may further be provided in a top, middle and bottom orientation, respectively, in a coaxial orientation, and with cylindrically aligned and removably attached exteriors such that the engagement and stirring functions of the test method may be provided. Additionally, and in an embodiment, attaching the encasing (10), the motor (5) and the clutch assembly (14) to the enclosing element (16) may allow the operator to seal vapor present in the container from the motor (5). The step of magnetically coupling the motor (5) and interchangeable rotationally configured stir implements (2) by way of a magnetic clutch mechanism may further serve to seal vapor present in the container from the motor (5). However, the primary means of sealing the container from the clutch may be performed by the step of removably attaching the encasing (10), the motor (5) and the clutch assembly (14) to the enclosing element.

Furthermore, in embodiments, the testing of soil samples may include steps to decontaminate the test device after stirring. This may include removing the encasing (10), the motor (5) and/or the clutch assembly (14) from the enclosing element. In other embodiments, this may also include at least partially opening the cylindrical container (1) by removing an enclosing element (16), removing the shaft element (3) and interchangeable rotationally configured stir implements (2) from the cylindrical container (1), and rinsing the a shaft element (3), plurality of interchangeable rotationally configured stir implements (2), and cylindrical container (1) prior to reuse of the soil stirring and testing device (15). Additional steps may be taken to decontaminate the motor (5), the clutch assembly (14), the encasing (10), and the enclosing element.

In an embodiment, the present invention may include regulating the speed of the motor (5) and the motion of the interchangeable rotationally configured stir implements (2), for example, by regulating a voltage applied to the motor (5). Accordingly, the present invention may include a speed regulator. This may allow the shaft element (3) and an interchangeable rotationally configured stir implement (2) to rotate at a constant speed of a shaft element (3). Regulating voltage, either alone or in combination with magnetically coupling the motor (5) to the interchangeable rotationally configured stir implements (2), can serve to maintain the coupling between the motor (5) and the interchangeable rotationally configured stir implements (2) and may assure proper stirring for extraction.

In an embodiment, the present invention may include a circuit which may be electrically and operationally connected to the motor (5). During motor operation, the circuit may serve to control the applied voltage or may limit the electrical current of the motor if the shaft and interchangeable rotationally configured stir implements encounter increased forces (e.g. torque) during stirring. A constant speed circuit may result in a relatively constant voltage that may ensure a proper mixing speed, and in embodiments, ensure coupling between the motor (5) and the interchangeable rotationally configured stir implements (2). This feature may serve not only to maintain the magnetic coupling provided by a magnetic clutch mechanism, but further may serve to provide a constant stirring motion and speed for consistent sample stirring and subsequent extraction.

Each of the described embodiments could include various facets of the present invention. Some may include rotation elements and rotative movements, while others may not include such elements. Some may include varieties of speed regulation. The market place and manufacturing concerns may dictate the appropriate embodiments for the present invention.

As can be easily understood from the foregoing, the basic concepts of the present invention may be embodied in a variety of ways. It involves both soil testing techniques as well as devices to accomplish the appropriate soil test element. In this application, the soil testing techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may be included for the device described, and also method or process claims may be included to address the functions the invention and each element performs. Neither the description nor the terminology is intended to limit the scope of the claims herein included.

It should also be understood that a variety of changes may be made without departing from the essence of the invention. Such changes are also implicitly included in the description. They still fall within the scope of this invention. A broad disclosure encompassing both the explicit embodiment(s) shown, the great variety of implicit alternative embodiments, and the broad methods or processes and the like are encompassed by this disclosure and may be relied upon for support of claims in this application. It should be understood that such language changes and broader or more detailed claiming may be accomplished at a later date (such as by any required deadline). With this understanding, the reader should be aware that this disclosure is to be understood to be designed to yield a patent covering numerous aspects of the invention both independently and as an overall system.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of an “enclosing element” should be understood to encompass disclosure of the act of “enclosing”—whether explicitly discussed or not—and, conversely, were there effectively disclosure of the act of “enclosing”, such a disclosure should be understood to encompass disclosure of an “enclosing element” and even a “means for enclosing.” Such changes and alternative terms are to be understood to be explicitly included in the description.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the list of references or other information statement filed with the application are hereby appended and hereby incorporated by reference, however, as to each of the above, to the extent that such information or statements incorporated by reference might be considered inconsistent with the patenting of this/these invention(s) such statements are expressly not to be considered as made by the applicant(s).

Thus, the applicant(s) should be understood to have support to claim and make a statement of invention to at least: i) each of the stirring devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) the various combinations and permutations of each of the elements disclosed, and xii) each potentially dependent claim or concept as a dependency on each and every one of the independent claims or concepts presented.

With regard to claims whether now or later presented for examination, it should be understood that for practical reasons and so as to avoid great expansion of the examination burden, the applicant may at any time present only initial claims or perhaps only initial claims with only initial dependencies. Support should be understood to exist to the degree required under new matter laws—including but not limited to European Patent Convention Article 123(2) and United States Patent Law 35 USC § 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible.

Finally, any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

What is claimed is:

1. A method for testing organic contaminants in soil comprising the steps of:
selecting a soil sample;
selecting an organic contaminant test substance suitable with said soil sample;
adding said organic contaminant test substance to said soil sample to create a soil testing sample;
putting said soil testing sample in said cylindrical container;
axially restraining a plurality of interchangeable rotationally configured stir implements with a shaft element in said cylindrical container;
detachably connecting said plurality of interchangeable rotationally configured stir implements to said shaft element;
providing a multi-directional restraint for said plurality of interchangeable rotationally configured stir implements;
enclosing said soil testing sample in said cylindrical container;
sealing said soil testing sample from said motor;
powering a motor to which said shaft element is responsive;
stirring said soil testing sample causing common axial displacement by said plurality of interchangeable rotationally configured stir implements to create an extraction sample; and
analyzing said extraction sample to determine the presence of an organic contaminant.

2. A method for testing organic contaminants in soil in claim 1 wherein said step of stirring said soil testing sample comprises the step of divergently displacing said soil testing sample in said cylindrical container with one of said interchangeable rotationally configured stir implements.

3. A method for testing organic contaminants in soil in claim 2 wherein said step of divergently displacing said soil testing sample comprises the step of divergently displacing said soil testing sample with an angularly displaced circular element.

4. A method for testing organic contaminants in soil in claim 2 wherein said step of divergently displacing said soil testing sample comprises the step of divergently displacing said soil testing sample with an angularly displaced linear element.

5. A method for testing organic contaminants in soil in claim 1 wherein said step of stirring said soil testing sample comprises the step of shearing said soil testing sample in said cylindrical container with one of said interchangeable rotationally configured stir implements.

6. A method for testing organic contaminants in soil in claim 5 wherein said step of shearing said soil testing sample comprises the step of shearing said soil testing sample with an angularly displaced circular element.

7. A method for testing organic contaminants in soil in claim 1 wherein said step of stirring said soil testing sample comprises the step of gently displacing said soil testing sample with one of said interchangeable rotationally configured stir implements.

8. A method for testing organic contaminants in soil in claim 1 wherein said step of stirring said soil testing sample comprises the step of gently displacing plant material with one of said interchangeable rotationally configured stir implements.

9. A method for testing organic contaminants in soil in claim 1 wherein said step of stirring said soil testing sample comprises the step of gently displacing stringy plant material with one of said interchangeable rotationally configured stir implements.

10. A method for testing organic contaminants in soil in claim 8 wherein said step of gently displacing plant material comprises the step of gently displacing stringy plant material with a round ended bar element.

11. A method for testing organic contaminants in soil in claim 1 wherein said step of stirring said soil testing sample comprises the step of displacing said soil testing sample having at least small rocks.

12. A method for testing organic contaminants in soil in claim 11 wherein said step of displacing said soil testing sample having at least some small rocks comprises displacing said soil testing sample with an angularly displaced linear element.

13. A method for testing organic contaminants in soil in claim 1 wherein said step of selecting said soil sample comprises the step of selecting a soil sample having at least small rocks.

14. A method for testing organic contaminants in soil in claim 1 wherein said step of selecting said soil sample comprises the step of selecting a soil sample having at least plant material.

15. A method for testing organic contaminants in soil in claim 1 wherein said step of selecting said soil sample comprises the step of selecting a soil sample having at least stringy plant material.

16. A method for testing organic contaminants in soil in claim 1 wherein said step of analyzing said extraction sample to determine the presence of said organic contaminant comprises the step of selecting an analysis element from the group consisting of UV absorption spectroscopy, visible absorption spectroscopy, fluorescence spectroscopy, raman spectroscopy, gas chromatography, mass spectrometry, atomic fluorescence, atomic absorption, atomic emission, electrochemistry, calorimetric reactions, titration, and gravimetric analysis.

17. A method for testing organic contaminants in soil in claim 1 wherein said step of selecting said organic contaminant test substance comprises the step of selecting said organic contaminant test substance from the group consisting of organic solvents and aqueous solvents.

18. A method for testing organic contaminants in soil in claim 1 wherein said step of selecting said organic contaminant test substance comprises the step of selecting said organic contaminant test substance from the group consisting of alcohols, aliphatic hydrocarbons, aromatic hydrocarbons, chlorinated solvents, ketones, esters, amines, ethers, sulfides, carboxylic acids, and surfactant solutions.

19. A method for testing organic contaminants in soil in claim 1 wherein said step of analyzing said extraction sample to determine the presence of said organic contaminant comprises the step of analyzing to determine said organic contaminant selected from the group consisting of diesel, tar, motor oil, fuel oil, petroleum, coal tar, gasoline, aviation gasoline, and synthetic motor oil.

20. A method for testing organic contaminants in soil in claim 1 wherein said step of axially restraining said plurality of interchangeable rotationally configured stir implements in said cylindrical container comprises the step of locating said interchangeable rotationally configured stir implements above said soil testing sample.

21. A method for testing organic contaminants in soil in claim 1 wherein said step of stirring said soil testing sample comprises the step of rotating said shaft element.

22. A method for testing organic contaminants in soil in claim 21 wherein said step of rotating said shaft element comprises the step of continuously rotating said shaft element.

23. A method for testing organic contaminants in soil in claim 1 and further comprising the step of magnetically coupling said shaft element to said motor.

24. A method for testing organic contaminants in soil in claim 1 wherein said step of sealing said soil testing sample from said motor comprises the step of magnetically sealing said soil testing sample from said motor.

25. A method for testing organic contaminants in soil in claim 1 and further comprising the steps of:
selecting a drying element; and
adding said drying element to said soil sample.

26. A method for testing organic contaminants in soil in claim 25 wherein said step of selecting said drying element and adding said drying element to said soil sample comprises the steps of selecting calcium oxide and adding said calcium oxide to said soil sample.

27. A method for testing organic contaminants in soil in claim 1 wherein said step of powering said motor comprises the step of regulating the speed of said stirring said soil testing sample.

28. A method for testing organic contaminants in soil in claim 1 wherein said step of powering said motor comprises the step of regulating a voltage to which said shaft element is responsive.

29. A method for testing organic contaminants in soil in claim 1 wherein said step of powering said motor comprises the step of powering said motor with a battery.

30. A method for testing organic contaminants in soil in claim 1 wherein said step of stirring said soil testing sample comprises the step of maintaining a constant stirring speed of said soil testing sample.

31. A method for testing organic contaminants in soil in claim 1 wherein said step of enclosing said soil testing sample in said cylindrical container comprises the step of enclosing said soil testing sample in said cylindrical container with a jar cover.

32. A method for testing organic contaminants in soil in claim 1 and further comprising the step of housing at least said motor in a cylindrical encasing.

33. A method for testing organic contaminants in soil in claim 32 and further comprising the step of cylindrically aligning an exterior of said cylindrical container, said enclosing element, and said cylindrical encasing.

34. A method for testing organic contaminants in soil in claim 1 wherein said step of selecting said soil sample comprises the step of screening said soil sample to remove larger items from said soil sample.

35. A method for testing organic contaminants in soil in claim 1 and further comprising the steps of:
removing an enclosing element from said cylindrical container;
removing said shaft element and plurality of interchangeable rotationally configured stir implements from said cylindrical container;
rinsing said shaft element, said plurality of interchangeable rotationally configured stir implements, and said cylindrical container; and
decontaminating at least said motor and said enclosing element.

36. An organic contaminant testing system for soil comprising:
a cylindrical container having an opening;
a soil sample;
an organic contaminant test substance;
a cylindrical removable enclosing element configured to cover said opening of said cylindrical container;
a shaft element through said cylindrical removable enclosing element;
a seal interposed adjacent to said shaft element and cylindrical removable enclosing element;
an interchangeable rotationally configured stir implement;
a multi-directional restraint to which said interchangeable rotationally configured stir implement is responsive;
a user detachable connection element connecting said interchangeable rotationally configured stir implement to said shaft element;
a motor to which said shaft element is responsive;
a power source to which said motor is responsive; and
an analysis element to test said soil sample for an organic contaminant.

37. An organic contaminant testing system for soil in claim 36 wherein said interchangeable rotationally configured stir implement is selected from the group consisting of a round ended bar element, an angularly displaced circular element, an angularly displaced linear element, a divergent displacer element; and a gentle displacer element.

38. An organic contaminant testing system for soil in claim 36 wherein said interchangeable rotationally configured stir implement and said soil sample are selected from the group consisting of:
a round ended bar element used with plant material,
round ended bar element used with stringy plant material,
a divergent displacer element used with small rocks,
a gentle displacer element used with plant material,
a gentle displacer element used with stringy plant material, and
an angularly displaced linear element used with small rocks.

39. An organic contaminant testing system for soil in claim 36 wherein said soil sample comprises at least small rocks.

40. An organic contaminant testing system for soil in claim 36 wherein said soil sample comprises at least plant material.

41. An organic contaminant testing system for soil in claim 36 wherein said soil sample comprises at least stringy plant material.

42. An organic contaminant testing system for soil in claim 36 wherein said analysis element is selected from the group consisting of UV absorbtion spectroscopy, visible absorption spectroscopy, fluorescence spectroscopy, raman spectroscopy, gas chromatography, mass spectrometry, atomic fluorescence, atomic absorption, atomic emission, electrochemistry, colorimetric reactions, titration, and gravimetric analysis.

43. An organic contaminant testing system for soil in claim 36 wherein said organic contaminant test substance is selected from the group consisting of organic solvents and aqueous solvents.

44. A method for testing organic contaminants in soil in claim 36 wherein said organic contaminant test substance is selected from the group consisting of alcohols, aliphatic hydrocarbons, aromatic hydrocarbons, chlorinated solvents, ketones, esters, amines, ethers, sulfides, carboxylic acids, and surfactant solutions.

45. An organic contaminant testing system for soil in claim 36 wherein said soil sample comprises at least fuel.

46. An organic contaminant testing system for soil in claim 45 wherein said fuel is selected from the group consisting of diesel, tar, motor oil, fuel oil, petroleum, coal tar, gasoline, aviation gasoline, and synthetic motor oil.

47. An organic contaminant testing system for soil in claim 36 wherein said interchangeable rotationally configured stir implements are located above soil sample.

48. An organic contaminant testing system for soil in claim 36 and further comprising a clutch assembly to which said shaft element is responsive.

49. An organic contaminant testing system for soil in claim 48 wherein said clutch assembly comprises a magnetic clutch assembly.

50. An organic contaminant testing system for soil in claim 49 wherein said magnetic clutch assembly comprises a seal between said cylindrical container and said motor.

51. An organic contaminant testing system for soil in claim 36 and further comprising a drying element.

52. An organic contaminant testing system for soil in claim 51 wherein said drying element comprises calcium oxide.

53. An organic contaminant testing system for soil in claim 36 and further comprising a speed regulator.

54. An organic contaminant testing system for soil in claim 36 and further comprising a circuit.

55. An organic contaminant testing system for soil in claim 36 wherein said power source comprises a battery.

56. An organic contaminant testing system for soil in claim 36 and further comprising a constant speed regulator of said shaft element and said interchangeable rotationally configured stir implements.

57. An organic contaminant testing system for soil in claim 36 wherein said cylindrical removable enclosing element comprises ajar cover.

58. An organic contaminant testing system for soil in claim 36 and further comprising a cylindrical encasing around said motor.

59. An organic contaminant testing system for soil in claim 58 wherein said cylindrical encasing around said motor is selected from the group consisting of a polypropylene encasing, glass encasing, polyphenylene sulfide encasing, polyethylene encasing, chlorofluorocarbon polymer encasing, teflon polymer encasing, and polyphthalamide encasing.

60. An organic contaminant testing system for soil in claim 36 wherein said cylindrical removable enclosing element is selected from the group consisting of a polypropylene element, glass element, polyphenylene sulfide element, polyethylene element, chlorofluorocarbon polymer element, teflon polymer element, and polyphthalamide element.

61. An organic contaminant testing system for soil in claim 36 wherein said cylindrical container is selected from the group consisting of polypropylene container, glass container, polyphenylene sulfide container, polyethylene container, chlorofluorocarbon polymer container, teflon polymer container, and polyphthalamide container.

62. An organic contaminant testing system for soil in claim 36 wherein said cylindrical removable enclosing element comprises an encasing for at least said motor.

63. An organic contaminant testing system for soil in claim 49 wherein said cylindrical removable enclosing element comprises an encasing for at least said motor and said magnetic clutch assembly.

64. An organic contaminant testing system for soil in claim 49 wherein said cylindrical removable enclosing element, said cylindrical container, and said magnetic clutch assembly comprises a cylindrically aligned exterior.

65. An organic contaminant testing system for soil in claim 49 wherein said motor, said magnetic clutch assembly, and said cylindrical container comprises a top, middle, and bottom arrangement, respectively.

66. An organic contaminant testing system for soil in claim. 49 wherein said cylindrical removable enclosing element, said magnetic clutch assembly, and said cylindrical container comprise a coaxial arrangement.

67. An organic contaminant testing system for soil in claim 63 wherein said encasing, said cylindrical removable enclosing element, and said cylindrical container comprises a cylindrically aligned exterior.
